(19) United States
(12) Patent Application Publication
(10) Pub. No.: US 2014/0235293 A1
(43) Pub. Date: Aug. 21, 2014

(54) Personal Health Monitoring System

(71) Applicant: Michael L. Sheldon, Georgetown, TX (US)

(72) Inventor: Michael L. Sheldon, Georgetown, TX (US)

(21) Appl. No.: 14/156,582

(22) Filed: Jan. 16, 2014

Related U.S. Application Data
(60) Provisional application No. 61/850,507, filed on Feb. 15, 2013.

(51) Int. Cl. A61B 5/00; H04M 1/21; H04M 1/02

(52) U.S. Cl. CPC A61B 5/0022; H04M 1/0202; H04M 1/21
USPC 455/556.1

(57) ABSTRACT

A personal health monitor device comprising memory for collecting and storing attributes from an individual and a processor for quantizing each attribute in such a way as to indicate a normal range for that attribute and for measuring deviations from that normal range. The processor further calculates the well-being of the individual using the deviations measured. The results are displayed indicating the well-being of the individual.

### CROSS-REFERENCE TO RELATED APPLICATION

This application claims the benefit under 35 U.S.C. §119(e) of U.S. Provisional Application Ser. No. 61/850,507, entitled “Personal Health Monitoring System,” filed Feb. 15, 2013, the entire disclosure of which is hereby expressly incorporated by reference herein.

### FIELD OF DISCLOSURE

The present disclosure relates generally to a personal health monitoring system and more particularly to the use of a device or system to monitor a person's physiological condition or attributes and use that information to diagnose and/or advise a user on his or her well-being.

### DISCUSSION OF RELATED ART

Systems for monitoring various physiological conditions for an individual are fairly common. One such system available today includes a wearable health monitoring system that includes sensors that are integrated with a telemedicine system. For this system, various sensors are attached to an individual and the sensed data that is created is communicated to a phone. Once collected at the phone the data is then sent to a remote server where doctors and trained physicians can analyze the data. Similar types of systems have also been used by athletes for measuring their physical attributes during training. Again, these systems collect the sensed data and then send the information to a tablet or computer to analyze it. Sometimes a phone is used to get the data to the tablet or computer. In these type of systems, performance is measured, not the health and well-being of the user.

What is needed is a health monitoring system that is integrated into a cellular phone or a tablet with cellular phone communications which allows a user to collect a wide variety of data including various physiological conditions to analyze for the purpose of determining the wellbeing of that user. Then should the need arise, the collected information, analysis, or other information could be sent to a treating physician using the cellular communication feature for further evaluation. This type of system would not only be convenient and practical, since everyone is currently using their cell phone or tablets for a variety of other application, but also beneficial because it would allow the user to maintain control and security over that personal individual data. As a result, great deal of expense in time and money could be saved by avoiding unnecessary doctor visits.

### SUMMARY

This invention relates to a personal health monitoring system for a cellular phone or tablet. The system includes sufficient memory capable of storing sensed data regarding attributes of an individual. It is preferred that the sensed data has a time stamp associated with it to identify when the data was collected. There is at least one well-being software application stored on the phone or tablet and selectable by a user for accessing the well-being of the user. Once selected, a processor that is capable of accessing the memory module for necessary sensed data to analyze the well-being of the individual in accordance with the well-being software application selected by the user provides results indicating the current well-being state of the user, which is displayed on the phone screen to the user.

### BRIEF DESCRIPTION OF THE DRAWINGS

FIG. 1 is a diagram of a personal health monitoring system capable of collecting and storing data representing a user's personal attributes from different sources for the purpose of analyzing the attributes using a processor to determine and display the well-being results of the user.

FIG. 2 illustrates a display of a phone or tablet showing functions that may be selected for monitoring the health of a user.

FIG. 3 shows an example of a series of health assessment questions that the monitoring device could query the user for to collect additional information regarding the user's weight and blood pressure and some calculated results such as body mass index and general assessment of blood pressure based on those answers.

FIG. 4 shows an example of a series of questions used to query the user regarding medical history of the user.

FIG. 5 illustrates another display of a phone or tablet showing functions that may be selected for monitoring the health of a user.

FIG. 6 shows a graph illustrating the measurements of an attribute such as blood pressure for the user over a 24 hours period.

FIG. 7 shows a graph illustrating the measurements of the base line and trend of an attribute such as blood pressure of the user over a period of 30 days.

FIG. 8 illustrates another display of a phone or tablet showing suggestions generated by the health monitoring system that the user may want to consider as a result of the historical analysis of the users blood pressure.

### DETAILED DESCRIPTION

The present invention relates to an individual health monitoring system that is preferably integrated into a cellular phone for personal use. While a cellular phone is shown and described as the preferred embodiment, a tablet or similar computing device that has cellular communications capabilities is contemplated as a part of the concept of this invention. According to the concept of this invention, the phone or tablet includes a large data base for storing all of the data collected from sensing devices used to collect physical attributes of the individual and software applications that can be called upon by the user to evaluate the collected data to determine the well-being of the individual. Results of the analysis, suggestions or recommendations for improving that individual's health would be displayed on the phone's or tablet's display screen. Depending on the results of the analysis, the user may select to send these results and/or data used to generate these result to a treating physician using standard features of a cellular communications available today.

Preferably the data is collected wirelessly from the sensing devices, but could be downloaded directly from the sensing devices using typical wired connections such as USB cables. The data base used for storing this information must be large enough for storing large amounts of data regarding the physical attributes of the individual and includes at least one, but preferably may include many such physical attributes as body temperature, blood pressure, humidity, ECG, breathing, blood sugar, heartbeat, administered medications, etc. For example, a suitable application of the phase change memory chip introduced by IBM in Jun 2011 which is 100 times faster than flash memory and capable of storing four times the amount of data would work well in this type of application. Preferably the data is stored in association with the time that the data was created to provide a time line for the data. This would allow for historical time lines of data to be evaluated as well as identify and notify the individual when fresh data is needed to properly analyze the well-being of the individual. It is also preferred that additional information could be stored to further aid in the analysis of a person's well-being by including information such as medical history including if possible family medical history and personal medical history. Additional information that is unique to the individual could also be entered by the individual to form a more complete data set. For example information on food consumption, types of food, exercise information, sleep information, weight, prescription/medication (current and past), etc. It would be obvious to one skilled in the art that this information would be formatted in such a way as to allow it to be easy accessed and read as necessary by an application analyzing the data as well as be fully searchable. For example, being able to search and review current and past prescriptions can be critically important to determine the compatibility of new medication. The system could be used in a continuous mode for collecting individual data or uploaded periodically from a sensing device which is capable of collecting and storing the information.

One of the obvious benefits of such a system is that the system could discover, query the individual, or automatically identify symptoms, rather than asking the individual to recognize the systems for himself when a well-being application is selected. Often times an individual doesn't understand or appreciate what symptoms that they should identify as important. Another advantage is that the system could effectively be operated as a personal doctor's aid by providing medical alerts or early detection of diseases or harmful conditions which could even include reactions to new medications. In general, the system would enable an individual to monitor his own health and only consult a doctor if the need exists. Many unnecessary doctor visits could be eliminated. Further, by centralizing all the data on a personal device such as a cellular phone or tablet, the information can be keep confidential, secure, and under the control of the individual. If that individual wishes to share that information, the cellular communications feature provides a convenient way to share information or data with a treating physician. For example, imagine a patient that has high blood pressure and his prescription is about to run out. Rather than making an appointment to get a refill, the patient monitors his own blood pressure with a device approved by his physician and then sends the data to the physician who can then approve the proper dosage and renew or change the prescription at the pharmacy directly. Both the physician and the patient save a considerable amount of time which results in cost savings to the patient. This is just one example of the utility and benefit of such a device/system described herein. One skilled in the art would appreciate many other such examples of savings of time and money in the health care industry that could be employed using such a device/system.

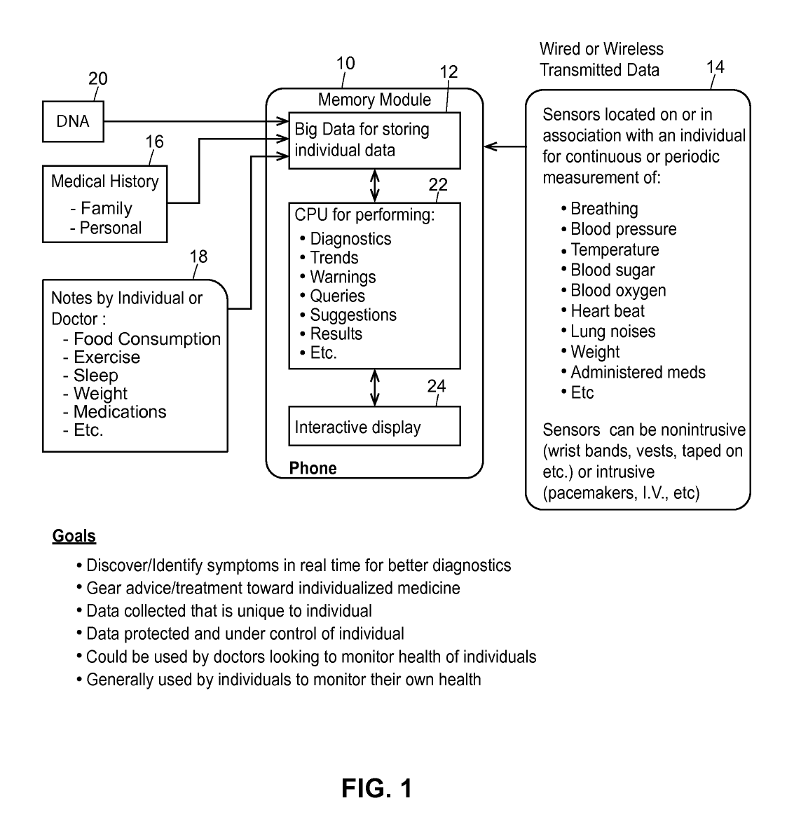

Referring now to the FIG. 1, a schematic diagram of a phone 10 is shown with data being communicated to it from various sources. As mentioned above, it is contemplated that a tablet or similar device could be used instead of a phone. It is preferred that such a device have a cellular communications feature to allow sharing the collected information with a treating physician. As shown, the phone 10 includes a high capacity memory module 12 for storing large amounts of individual data. Preferably the data is time stamped or references a time when the data was actually collected. While FIG. 1 shows a memory module for storing the individual data, one skilled in the art would appreciate that this data could be stored using a flash card, Sims card, or remotely such as using the Cloud to store data using conventional cellular technology and then called upon by the software application selected by the user to evaluate the collected data to determine the well-being of the individual. There are all kinds of data compression techniques available that could also be used to aid in the storage of such large amounts of data and are well known by those skilled in the art. For example, loss less compression and lossy less compression techniques can be used and have long been used by those skilled in the art to avoid storing unnecessary information and thereby increasing the capacity of the memory available. There are other techniques to help in extrapolating data when data is missing in the time line and may be used as an aid to analyze the current data for the well-being of the individual.

Data is collected from at least one senor and preferably several sensors 14 used to collect data in association with the physiological condition or attributes of the individual. For example, breathing, blood pressure, temperature, cholesterol, blood sugar, blood oxygen, heart beats, lung noises, weight, administered medications, etc. just to name a few. The sensors can be located on or in association with the individual user by way of a vest, armband, wrist band, ankle band, etc. Such devices are readily available. For example, Best Buy currently advertises and sells a host of wireless devices that sense and monitor individual physiological conditions. One such device is a wireless activity and sleep tracker. Another device is a “BodyMedia—Fit Link Armband” that measures calories burned, body temperature, steps and sweat, sleep quality, etc. and is wireless. Still others sell monitors for measuring blood pressure and blood sugar. The sensors could also be intrusive to the individual such as pace makers or other devices which deliver medication to the individual. In the preferred embodiment the data is collected by the sensing device and transmitted wirelessly to the phone's data storage module. However, data may be communicated from the devices to the data module in the phone via wires, such as a USB cable. In each case, time associated with when the data is collected is stored.

Since each individual is unique, relevant data from other sources are also preferred. As shown in FIG. 1, data including medical history for both family and personal 16 are also preferred. Additional information by the individual may also be provided. For example, information regarding the individual's food consumption, exercise routine, sleep habits, weight, medications, etc. 18 may also be provided. DNA information 20 may also be important information in the future for properly analyzing the data and could be included. Taking samples of bacteria from different areas of the body for analysis is also contemplated. As well-being programs are developed, one skilled in the art would realize that other data may be needed and collected.

In the preferred embodiment, the processor 22 of the phone 10 (or tablet not shown) is used to initiate applications which access the data from the memory module or the Cloud and perform calculations that are used by a variety of health or well-being programs. However, it is possible for a second processor to be provided and dedicated to these applications. As an example of the type of applications that could be provided, one could include a general diagnostics application on the well-being of the individual's blood pressure to determine if there may be issues of high blood pressure indicative of heart attacks, strokes, heart failure, kidney disease, stress, etc. Similarly an application could be provided to assess the individual's blood sugar to identify issues with diabetes. Since your blood sugar can fluctuate throughout the day, understanding your sugar levels over time will be important. Trends regarding various other health conditions such as good and bad cholesterol could also be performed. General health assessments, warnings, suggestions, and recommendations are a few of the benefits of these applications. Numerous other medical applications to assess the individual's well-being are possible but not mentioned here, because it would be obvious to one skilled the art that many such applications could be developed and downloaded on the phone or tablet and used in the manner described above. The results of these analysis and interaction with these applications can be shown on the interactive display 24 available on the phone or tablet (not shown) and in many useful and creative ways. Graphs showing trends or tables showing data are possible. Colorful alerts, warnings or suggestions may also be displayed. Further, forms can be created and displayed for querying the individual for more information to complete the data set for analysis. It should become clear that the concept of this invention contemplates storing and allowing the user to access many different expert medical applications which leverages the wealth of medical knowledge to evaluate and diagnose the data to determine the well-being of the individual.

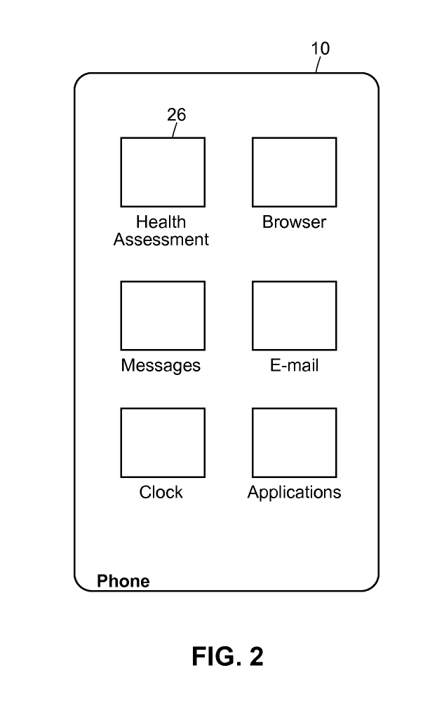

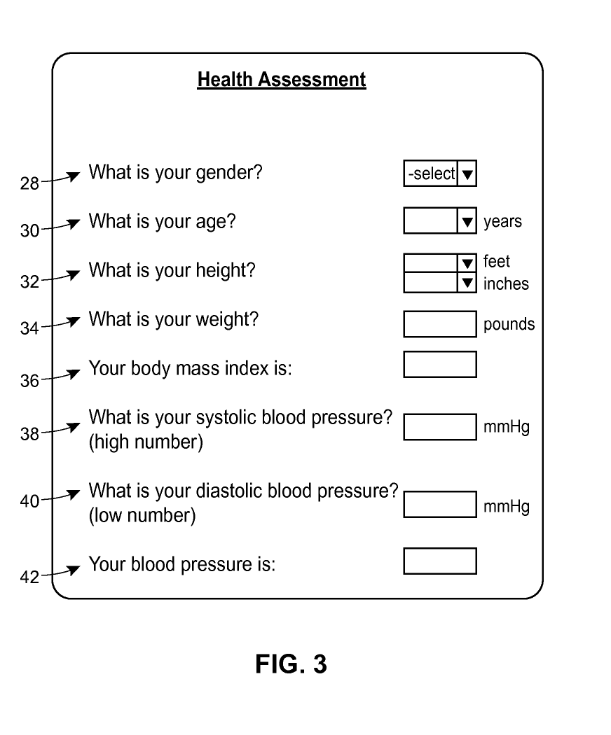

Referring to FIGS. 2-7, some screen shots of a cellular phone is shown and is used to illustrate how a user might select one of several potential health assessment applications that could be downloaded and stored on his phone. One skilled in the art would understand that the example provided is only an illustration of one the types of applications that could be stored and selected by a user. A host of other applications could be created, stored, and available to the user. As one example of an application that could be stored and selected, FIG. 2 shows a general health assessment program 26 using the interactive screen of his phone. Selecting this application could result in a query of information from the user. As shown and illustrated in FIG. 3 that could include the user's gender 28, age 30, height 32, and weight 34. This information could then be used to calculate the user's body mass index 36. Additional information could be queried such as blood pressure including the systolic and diastolic values 38 & 40. Based on this information, a health indication 42 can be automatically displayed as high, normal or low blood pressure to indicate the well-being of an individual. While this example demonstrates how a user would be queried to enter information regarding his blood pressure measured results, this information could be transmitted to the phone wirelessly or by wire from the measuring device as suggested above. As already mentioned and appreciated by one skilled in the art, a tablet or like device could be used in place of the phone for performing the general health assessment.

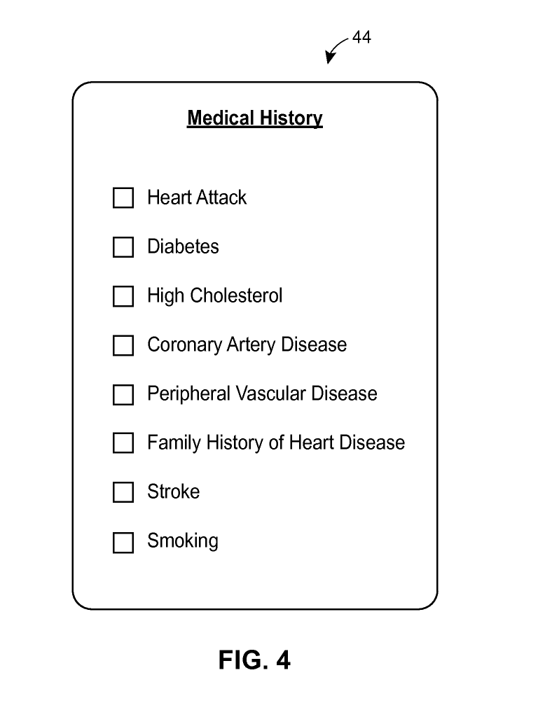

Referring now to FIG. 4, quires could also be made for medical history. As an example, a form 44 is shown asking the user to provide information on his medical history by interactively placing a check the boxes for the appropriate items shown. One skilled in the art would appreciate that there is an endless amount of medical history information that could be obtain from the user to help in the medical assessment of the well-being of the user. These forms could arranged and appear in an endless variety of ways. The present form shows boxes that would allow the user to select past medical issues or current conditions that would apply to that individual, such as previous heart attacks, diabetes, high cholesterol, coronary artery disease, peripheral vascular disease, family history of heart disease, stroke, and smoking just to name a few that are possible.

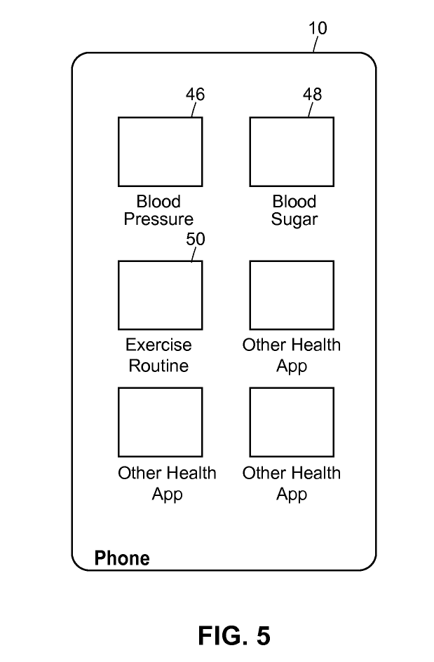

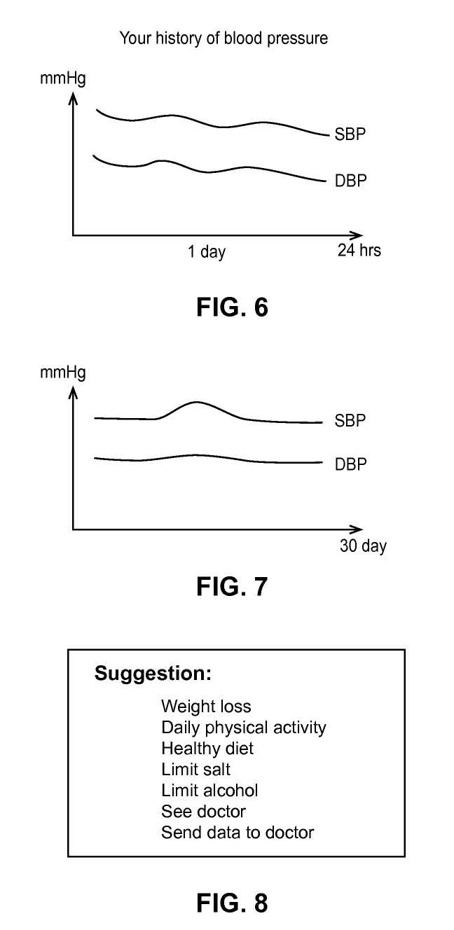

Turning the attention of the reader to FIG. 5, the user could select applications from his main menu shown of his phone 10 shown in FIG. 2 to display a variety of health monitor applications such as blood pressure 46, blood sugar 48, exercise routine 50, and others as shown. As one skilled in the art would appreciate, there are a variety of health monitor applications that could be created, stored and selected by the user. For example, the user could select the blood pressure application. Based on the historical information on measured blood pressure a graph could be displayed showing the user his blood pressure over the last 24 hours as illustrated in FIG. 6, over the last 30 days as illustrated in FIG. 7, or over any other desired range. Rather than showing a graph, tables or other ways of showing this information are possible and would be appreciated by one skilled in the art. Depending on the results of the blood pressure data, it is preferred that suggestions are provided to the user for improving the results of the users blood pressure as illustrated in FIG. 8. This could include such things as a recommendation that the user lose some weight, increase his daily physical activity, improve his diet, limit his salt or alcohol intake or see a doctor regarding his blood pressure. A variety of other suggestions or recommendations is possible and would be realized by one skilled in the art depending on the data collected and analyzed. Further, the health monitor application could further refine these recommendation or suggestions by using the historical data collected from other measured physiological attributes of the individual. For example, the application could eliminate some of these suggestions or recommendations to one or two for the user to consider and follow. Therein lays one of the benefits of this concept. By collecting data uniquely from one individual, the recommendations or suggestion can be tailored, based on that data, to the needs of that individual. If the only recommendation or suggestion is to see your physician, then the information that resulted in this conclusion could be sent to the physician using the phone features common to most cellular phones.

While the invention has been particularly shown and described with reference to a preferred embodiment, it will be understood by those skilled in the art that various changes in form and detail may be made therein without departing from the spirit and scope of the invention.

What is claimed is:

1. A personal health monitoring system for a cellular phone comprising:
a memory module capable of storing sensed data regarding attributes of an individual, wherein the sensed data has a time stamp associated with it to identify when the data was collected
at least one well-being software application selectable by a user for accessing the well-being of the user,
a processor that is capable of accessing the memory module for necessary sensed data to analyze the well-being of the individual in accordance with the well-being software application selected by the user and providing results indicating the current well-being state of the user, and
a display for displaying the results of the analysis to the user.

2. The personal health monitoring system of claim 1 wherein the memory module uses cloud technology to store the data.

3. The personal health monitoring system of claim 1 wherein the memory module is located in the phone.

4. The personal health monitoring system of claim 1 wherein the well-being software applications are stored and selected to operate on the cellular phone.

5. The personal health monitoring system of claim 1 wherein the well-being software applications include measuring and monitoring blood pressure.

6. The personal health monitoring system of claim 1 wherein the well-being software applications include measuring and monitoring cholesterol.

7. A cellular phone well-being monitoring system for an individual wherein the cellular phone comprising:
at least one sensor for detecting the physical attributes of an individual,
a memory module for storing the physical attributes,
a well-being software application accessible by a user to evaluate the physical attributes stored in the memory to determine the well-being of the cellular user.

8. A method for monitoring the well-being of an individual using a cellular phone, comprising the steps:
collecting data from at least one sensor detecting the physical attributes of an individual in a memory module of a cellular phone,
storing at least one well-being software application in the memory module of the cellular phone,
running the software application using the collected data in the cellular phone to analysis and determine the well-being of an individual, and
displaying the well-being results of the individual.

9. A personal health monitoring device comprising:
a memory module capable of storing sensed data regarding attributes of an individual, wherein the sensed data has a time stamp associated with it to identify when the data was collected,
at least one well-being software application selectable by a user for accessing the well-being of the user,
a processor that is capable of accessing the memory module for necessary sensed data to analyze the well-being of the individual in accordance with the well-being software application selected by the user and providing results indicating the current well-being state of the user, and
a display for displaying the results of the analysis to the user.

10. The personal health monitoring device of claim 9 further includes cellular communications.

11. The personal health monitoring device of claim 9 wherein the device is a cellular phone

12. The personal health monitoring device of claim 9 wherein the device is a tablet.
